(19) United States
(12) Patent Application Publication
(10) Pub. No.: US 2014/0255526 A1
(43) Pub. Date: Sep. 11, 2014

(54) USE OF A GINSENG EXTRACT TO PREVENT AND TREAT ALOPECIA AREATA

(71) Applicant: AFEXA LIFE SCIENCES INC., Montreal (CA)

(72) Inventors: Jacqueline Shan, Edmonton (CA); Sandra Miller, St. Lambert (CA); Punithavathi Durairaj, Montreal (CA)

(21) Appl. No.: 14/353,231

(22) PCT Filed: Oct. 26, 2012
(86) PCT No.: PCT/CA2012/050765

§ 371 Date: Apr. 21, 2014

Related U.S. Application Data
(60) Provisional application No. 61/552,916, filed on Oct. 28, 2011.

(51) Int. Cl. A61K 36/258; A61K 31/7004; A61K 31/704

(52) U.S. Cl. CPC A61K 36/258; A61K 31/704; A61K 31/7004
USPC 424/728

(57) ABSTRACT

Ginseng extracts as well as preparations such as medicaments, pharmaceuticals and food or dietary supplements for use to treat or prevent alopecia areata in subjects requiring such treatment or prevention are provided. The ginseng extract is a carbohydrate extract that contains between about 60-70 wt % carbohydrates, for example.

### FIELD OF INVENTION

The invention relates to the field of alopecia and in particular to ginseng extracts for treating and preventing alopecia areata.

### BACKGROUND OF INVENTION

Alopecia areata (AA) is a disorder characterized by non-scarring hair loss. It can affect parts of the scalp (patchy AA), the entire scalp (alopecia totalis, AT), or the entire body (alopecia universalis, AU). It is estimated that AA affects 0.1 to 0.2% of the population in the US, with males and females equally affected. Paediatric AA accounts for approximately 20% of all cases. Up to 60% of patients with AA will experience their first hair loss incident before 20 years old and most patients will have more than one episode of hair loss. AA is an unpredictable disease; up to 50% of patients will recover within 1 year without treatment, but will often relapse. Approximately 5% of cases will progress to AT/AU, from which the chance of full recovery is less than 10% [2]. Additionally, due to the social stigma of hair loss, alopecia areata can have severe psychological impacts, especially for children [4]. It has been suggested that there is a high incidence of anxiety, depression, and other psychological effects in AA patients [2].

AA is an autoimmune disease which targets the hair follicles [4]. Normally, hair follicles are an immunoprivileged site during the growth stage of the hair cycle (or anagen phase). The cause of the AA is unknown, but several hypotheses have been proposed. Without wishing to be bound by theory, the most prevalent suggestion is that, following the loss of immune privilege, there is a T-cell-mediated attack on hair follicles in the anagen phase [4]. It has been proposed that this chain of events occurs in genetically predisposed individuals who are also influenced by environmental factors, such as stress or diet [2], for example.

Various therapeutic agents have been described for the treatment of alopecia areata, but none cure the disease or prevent relapses [3]. The commonly used treatment include corticosteroids, topical immunotherapy (also known as topical sensitizers), psoralen combined with ultraviolet A phototherapy (PUVA), minoxidil, dithranol (also known as anthralin), and immunosuppressants (such as cyclosporine) [3]. The initial effectiveness of current treatment options ranges from less than 10% up to approximately 60%. However, none of the common treatment options have shown a significant long-term benefit compared with placebo [3]. In addition, all of the treatments have documented side effects which range from being uncomfortable (e.g. dermatitis and folliculitis) to serious (e.g. increased risk of skin cancer and Cushing Syndrome) [3]. Given the adverse side effects and ineffectiveness, many patients may view the current treatment options as unacceptable.

Ginseng has a long history of use as a medicinal herb in traditional medicine in Asia and North America. It has been shown to have a range of medicinal properties, but without many of the adverse side effects associated with traditional pharmaceuticals. The monograph for Panax quinquefolius (North American ginseng) indicates that there are no known contraindications or adverse effects [1].

CVT-E002 (previously described in U.S. Pat. No. 6,432,454) is a carbohydrate-rich extract of North American ginseng (Panax quinquefolius). CVT-E002 comprises 60-70 wt % carbohydrates and the carbohydrate content comprises about 0.5-5 mol % rhamnose, about 11-22 mol % galacturonic acid, about 40-60 mol % glucose, about 10-19 mol % galactose and about 11-19 mol % arabinose.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide ginseng extracts for use to treat or prevent alopecia areata. In accordance with one aspect of the invention, there is provided a method of preventing or treating alopecia areata in a subject in need of such treatment, the method comprising administrating to the subject an effective amount of a ginseng extract having a carbohydrate content comprising about 0.2-5.5 mol % rhamnose, about 9-25 mol % galacturonic acid, about 35-65 mol % glucose, about 8-22 mol % galactose, and about 8-22 mol % arabinose.

In accordance with another aspect of the invention, there is provided a use of a ginseng extract to treat or prevent alopecia areata in a subject in need of such treatment, wherein the ginseng extract has a carbohydrate content comprising about 0.2-5.5 mol % rhamnose, about 9-25 mol % galacturonic acid, about 35-65 mol % glucose, about 8-22 mol % galactose, and about 8-22 mol % arabinose.

In accordance with another aspect of the invention, there is provided a use of a ginseng extract in the preparation of a medicament for preventing or treating alopecia areata, wherein the ginseng extract has a carbohydrate content comprising about 0.2-5.5 mol % rhamnose, about 9-25 mol % galacturonic acid, about 35-65 mol % glucose, about 8-22 mol % galactose, and about 8-22 mol % arabinose.

In accordance with another aspect of the invention, there is provided a method of preventing or treating alopecia areata in a subject in need of such treatment comprising administrating to the subject a ginseng extract comprising ginsenosides Rg1, Re, Rb1, Rd and Rc and from about 60 to about 70% (w/w) carbohydrates.

In accordance with another aspect of the invention, there is provided a use of a ginseng extract comprising ginsenosides Rg1, Re, Rb1, Rd and Rc and from about 60 to about 70% (w/w) carbohydrates for preventing or treating alopecia areata in a subject in need of such treatment.

In accordance with another aspect of the invention, there is provided a use of a ginseng extract comprising ginsenosides Rg1, Re, Rb1, Rd and Rc and from about 60 to about 70% (w/w) carbohydrates in the preparation of a medicament for preventing or treating alopecia areata.

### BRIEF DESCRIPTION OF THE DRAWINGS

FIG. 1 shows the effects of a daily 80 mg dose of CVT-E002 on a C3H/HeJ mouse that has developed alopecia areata. This is a representative example of the 14 mice in the treatment group of the treatment study.

### DETAILED DESCRIPTION OF THE INVENTION

The present invention relates to treating, preventing, or treating and preventing alopecia areata using one or more than one ginseng extract. In various embodiments, the one or more ginseng extracts may be rich in carbohydrates, comprise a composition comprising carbohydrate rich extracts of ginseng, or be the ginseng extract CVT-E002. Furthermore, the present invention relates to compositions comprising one or more than one ginseng extract, carbohydrate rich ginseng extracts, or the extract CVT-E002 for the treatment and/or prevention of alopecia areata.

To facilitate the understanding of the invention, the following definitions are provided.

“Biocompatible” means a compound or mixture of compounds that does not generate a significant undesirable host response for the intended utility. Biocompatible materials are typically non-toxic for the intended utility. For human utility, a biocompatible compound or mixture of compounds is most preferably non-toxic to humans or human tissues.

“Carrier” means a suitable vehicle which is biocompatible and/or pharmaceutically acceptable, including for instance, one or more solid, semisolid or liquid diluents, excipients, adjuvants, flavours, or encapsulating substances which are suitable for administration.

An “extract” refers to a preparation obtained following extraction of a plant or plant part with a suitable solvent such as, for example, water, ethanol, a mixture thereof, oils or other suitable solvent well known in the state of the art of plant extraction. The extract can be used as such if it is pharmacologically acceptable (i.e., having pharmacological activity for the intended purpose), or the solvent of the resulting solution(s) may be removed and the residue used as such or after further work up, for example, after further purification, blending with other active ingredients, adding a carrier, or re-suspending in a suitable solvent. The term “plant” is understood to mean the whole plant or one or more plant parts, for example, the leaves, the stems, the fruits or roots, comprising one or more active ingredients.

“Ginseng” is meant to refer to a plant, plant species or extract comprising ginsenosides, for example the ginsenosides Rg1, Re, Rb1, Rd and Rc. For example, ginseng may refer to plants belonging to Araliaceae that comprise ginsenosides, for example Panax species, and may include, but not be limited to, those Panax species listed in Table 1. It will be understood by those skilled in the art that there may be other Panax species belonging to Araliaceae which may be used within the context of the present invention that are not listed in Table 1. The term “ginseng” also includes wild, cultivated, or processed ginseng. Wild ginseng is ginseng which has not been planted and cultivated domestically, but grows naturally and is harvested from wherever it is found to be growing. Processed ginseng includes, for example, fresh or green ginseng, white ginseng, and red ginseng. Fresh or green ginseng is raw ginseng harvested in the field. White ginseng is obtained by drying fresh ginseng, and red ginseng is obtained by steaming fresh ginseng followed by drying the steamed ginseng.

TABLE 1
Examples of Varieties and Types of Ginseng

| Latin name(s) | Common name(s) |
|---|---|
| Panax quinquefolius | North American/Canadian |
| Panax trifolia | Eastern region of North America |
| Panax ginseng C.A Meyer | Asian ginseng |
| Panax schinseng | Chinese ginseng |
| Panax japonicus | Japanese ginseng |
| Panax notoginseng | Korean ginseng |
| Panax pseudoginseng | Oriental ginseng |
| Panax vietnamensis | Vietnamese ginseng |
| Panax elegatior | Nepalese ginseng |
| Panax wangianus | Wild ginseng |
| Panax bipinratifidus | Green or fresh ginseng |
| | Red ginseng |
| | White ginseng |
| | Xi Yang Shen |
| | Ren Shen/Gao Li Shen |
| | Tienchi/Sanchi |
| | Sâm Ng [symbol] oc Linh |

A “ginseng extract” is meant to refer to an extract made from any variety and type of ginseng as listed in Table 1 or described above, and subfractions obtained from these ginseng extracts. In certain embodiments, the ginseng extract may be obtained from a Panax species, for example, North American ginseng (Panax quinquefolius). It will be appreciated by those skilled in the art that extracts from plants or plant parts other than ginseng, or synthetic extracts, comprising ginsenosides, for example the ginsenosides Rg1, Re, Rb1, Rd and Rc, may be considered ginseng extracts, and may equally well be used as defined herein provided that their chemical properties and activities are sufficiently similar to the ginseng extract described herein.

“CVT-E002” is meant to refer to an exemplary ginseng extract from Panax quinquefolius, which has been described in U.S. Pat. Nos. 6,432,454; 7,067,160; 7,186,423 and 7,413,756.

“Subject” means a human or other mammal including for example cows, sheep, horses, buffalo, lama, dogs, cats, rabbits, hamsters and guinea pigs.

“Patient” means a subject with a condition requiring treatment.

“Prevention” refers to reducing the incidence of alopecia areata episodes within a subject.

“Treatment” refers to reducing the severity and/or duration of the alopecia areata episodes within a subject.

As used herein, the term “about” refers to an approximately +/−10% variation from a given value. It is to be understood that such a variation is always included in any given value provided herein, whether or not it is specifically referred to.

As demonstrated herein, the carbohydrate rich ginseng extract, CVT-E002, has preventative and curative properties for alopecia areata. Thus, in certain embodiments, the present invention relates to a method of preventing or treating alopecia areata in a subject in need of such treatment, the method comprising administering to the subject an effective amount of a carbohydrate rich ginseng extract.

In certain embodiments, the present invention relates to the use of a carbohydrate rich ginseng extract in the preparation of a medicament for prevention or treatment of alopecia areata. The ginseng extract may be formulated as a pharmaceutical composition comprising a pharmaceutically acceptable carrier. Pharmaceutically acceptable carriers are well known to those skilled in the art. A non-limiting example of a carbohydrate rich ginseng extract is the ginseng extract CVT-E002.

Compositions and medicaments comprising one or more carbohydrate rich ginseng extracts may be administered by methods well known to those skilled in the art. Examples of pharmaceutical compositions include, but are not limited to, tablets, capsules, liquids, lozenges, lotions, aerosol, and solutions suitable for various routes of administration including, but not limited to, orally, via injection or infusion, intraperitoneally, topically, nasally, ocularly, vaginally or rectally, in solid, semi-solid or liquid dosage forms as appropriate and in unit dosage forms suitable for easy administration of fixed dosages. For example, in certain embodiments, a topical cream, lotion or gel comprising one or more carbohydrate rich ginseng extracts may be applied topically to prevent or treat alopecia areata. In some embodiments, the one or more carbohydrate rich ginseng extracts may be formulated for oral administration to prevent or treat alopecia areata.

In some embodiments, the ginseng extract may be prepared with carriers that will protect the active agents against rapid elimination from the body, such as a controlled release formulation, including implants, coatings and microencapsulated delivery systems.

In some embodiments, the present invention provides a food, nutraceutical or supplement (for example, a dietary supplement) comprising one or more carbohydrate rich extracts of ginseng. The food item, nutraceutical or supplement may be administered to a patient having alopecia areata, in an amount suitable to prevent, treat, or prevent and treat alopecia areata. The carbohydrate rich extract may be the exemplary ginseng extract CVT-E002.

In some embodiments, the carbohydrate rich ginseng extract comprises ginsenosides Rg1, Re, Rb1, Rd and Rc and from about 60 to about 70% (w/w) carbohydrates.

In certain embodiments, the carbohydrate rich ginseng extract may be CVT-E002 (previously described in U.S. Pat. No. 6,432,454). CVT-E002 is prepared from American ginseng (Panax quinquefolius) and comprises 60-70 wt % carbohydrates and the carbohydrate content comprises about 0.5-5 mol % rhamnose, about 11-22 mol % galacturonic acid, about 40-60 mol % glucose, about 10-19 mol % galactose and about 11-19 mol % arabinose.

CVT-E002 can be prepared as described in U.S. Pat. No. 6,432,454. Briefly, milled raw ginseng is treated with ethanol in an ethanol to ginseng ratio between about 8:1 to 16:1 by weight. The resulting slurry is heated to or near reflux for about 1-5 hours. After the reflux period, the mixture is separated into the supernatant and residue. The supernatant is discarded. The residue is treated with water (about 10-20 times by weight). The resulting slurry is heated to or near refluxing temperatures for a period of 1-3 hours. After the extraction is complete the mixture is separated into the supernatant (aqueous extract I) and a residue. The aqueous extract may be dried to give a carbohydrate rich ginseng extract. A method of preparing the exemplary ginseng extract CVT-E002 is provided in Example 1.

Alternatively, ginseng extracts prepared by other methods known to a person having ordinary skill in the art may equally well be used in the present context and are within the scope of the present invention, as long as their chemical properties and biological activities are sufficiently similar to the ginseng extract used herein. In certain embodiments, therefore, the carbohydrate rich ginseng extract is an extract that is substantially identical to the exemplary extract CVT-E002. By “substantially identical to CVT-E002” it is meant that the carbohydrate rich ginseng extract has alopecia areata preventing and/or treating activity and comprises about 55-75 wt % carbohydrates, for example between 60-70 wt %, and the carbohydrate content comprises about 0.2-5.5 mol % rhamnose, for example, about 0.4-5.5 mol %, about 0.4-5.2 mol % or about 0.5-5 mol % rhamnose; about 9-25 mol % galacturonic acid, for example, about 10-23 mol %, about 10-22 mol % or about 11-22 mol % galacturonic acid; about 35-65 mol % glucose, for example about 37-63 mol %, about 38-62 mol % or about 40-60 mol % glucose; about 8-22 mol % galactose, for example about 9-21 mol %, about 10-20 mol % or about 10-19 mol % galactose, and about 8-22 mol % arabinose, for example about 9-21 mol %, about 10-20 mol % or about 11-19 mol % arabinose.

In certain embodiments, an extract that is substantially identical to CVT-E002 may further comprise ginsenosides Rg1, Re, Rb1, Rd and Rc. In some embodiments, an extract that is substantially identical to CVT-E002 may comprise ginsenosides Rg1, Re, Rb1, Rd and Rc and from about 60 to about 70% (w/w) carbohydrates. In some embodiments, an extract that is substantially identical to CVT-E002 may comprise ginsenosides Rg1, Re, Rb1, Rd and Rc, and from about 60 to about 70% (w/w) carbohydrates and the carbohydrate content comprises about 0.2-5.5 mol % rhamnose, for example, about 0.4-5.5 mol %, about 0.4-5.2 mol % or about 0.5-5 mol % rhamnose; about 9-25 mol % gapacturonic acid, for example, about 10-23 mol %, about 10-22 mol % or about 11-22 mol % galacturonic acid; about 35-65 mol % glucose, for example about 37-63 mol %, about 38-62 mol % or about 40-60 mol % glucose; about 8-22 mol % galactose, for example about 9-21 mol %, about 10-20 mol % or about 10-19 mol % galactose, and about 8-22 mol % arabinose, for example about 9-21 mol %, about 10-20 mol % or about 11-19 mol % arabinose.

Such substantially identical extracts may be prepared from Panax quinquefolium or from other Panax species, such as those listed in Table 1, using standard methods known in the art.

An extract may be evaluated to assess and confirm an alopecia areata preventative, curative, or both preventative and curative property by conducting one or more in vitro or in vivo evaluations. Methods for testing for alopecia areata preventative, curative, or both preventative and curative properties are well known in the art and include, for example, the use of animal models such as those described in the Examples.

In some embodiments, the ginsenoside and carbohydrate content of extracts may be standardized for both chemical content and biological activity by chemical and biological fingerprinting methods. An exemplary chemical and biological fingerprinting method, ChemBioPrint®, has been described in U.S. Pat. No. 6,156,291. Other chemical and biological fingerprinting methods which are well accepted in the art are within the scope of the present invention.

Dosages of the ginseng extract in accordance with the invention depend upon the particular condition to be treated, as well as the age, sex and general health condition of the patient. However, suitable dosages may be found in the range between 1 and 1000 mg/kg body weight per day, or any amount therebetween, with between 1 and 10 daily doses. For example, the dosage may be between 1 and 500 mg/kg body weight per day, or any amount therebetween, between 1 and 250 mg/kg body weight per day, or any amount therebetween, between 1 and 200 mg/kg body weight per day, or any amount therebetween, between 1 and 150 mg/kg body weight per day, or any amount therebetween, or about 1, 10, 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000 mg/kg body weight per day, or any amount therebetween, with between 1, 2, 3, 4, 5, 6, 7, 8, 9, 10 daily doses.

In certain embodiments, the dosage of the extract is about 10-1000 mg/kg body weight, for example between about 50-1000 mg/kg body weight or between about 100-1000 mg/kg body weight.

Certain embodiments of the invention relate to the use of the carbohydrate rich ginseng extract(s) in combination with one or more conventional alopecia areata therapies. In some embodiments, the ginseng extracts may mitigate one or more side effect associated with the conventional therapy and/or may allow lower dosages of the conventional therapy to be used.

To gain a better understanding of the invention described herein, the following examples are set forth. It will be understood that these examples are intended to describe illustrative embodiments of the invention and are not intended to limit the scope of the invention in any way.

### EXAMPLES

### Example 1

### A Process for Preparing and Purifying Fraction CVT-E002

American ginseng root was chemically extracted and purified sequentially to give fractions CVT-E001 and CVT-E002 as described in U.S. Pat. No. 6,432,454. An amount of CVT-E002 was further purified to give fractions G₁, G₂, and G₃. The detailed description of the procedure is as follows.

One thousand grams of dried ground root of American ginseng was extracted with 8-12 liters of 85% ethanol on a water bath at 95-100° C. while stirring for 3 hours and filtered to give an alcohol solution and residue. The residue was combined with water (1:8-1:12) on a hot water bath with continuing agitation for 3 hours. After cooling to room temperature, the mixture was filtered. The filtrate was centrifuged at 5000 rpm for 10 min. The supernatant was concentrated and freeze dried to give extract CVT-E002. The amount of CVT-E002 produced by this method is approximately 10%-20% of the weight of the original raw ginseng.

### Example 2

### Prevention of Alopecia Areata in the C3H/HeJ Mouse Model

The C3H/HeJ mouse is a well-established model for alopecia areata in humans [2;5]. AA occurs spontaneously in C3H/HeJ mice and has several features that are clinically and pathologically similar to the human disease [5]. Through selective breeding, the incidence of AA in C3H/HeJ mice has been increased from 0.035%-0.25% to close to 20% [5]. The C3H/HeJ animal model provides a system to develop, test, and analyze new forms of treatment for AA [2].

C3H/HeJ mice were separated into two groups: control group (which had no CVT-E002 in diet) and treatment group (which were fed 80 mg/day of CVT-E002). The CVT-E002 was combined with chow and fed to the mice in the treatment group; while chow without CVT-E002 was fed to the control group. In the control group, 25% of mice developed alopecia, which is the percentage of C3H/HeJ mice that are expected to develop alopecia areata. None of the mice in the treatment group lost any fur.

### Example 3

### Treatment of Alopecia Areata in the C3H/HeJ Mouse Model

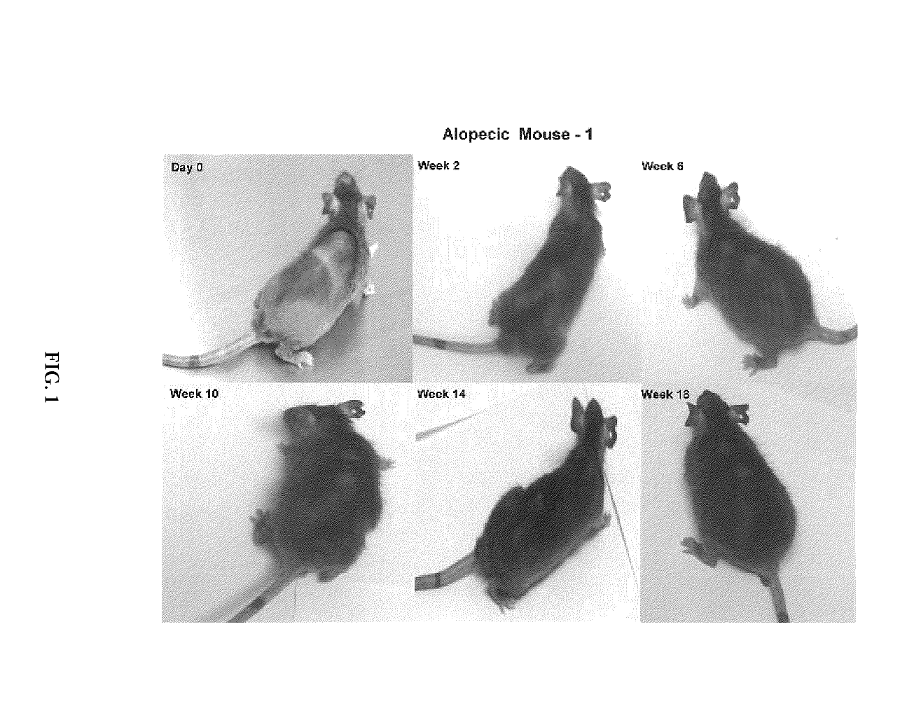

In another study with a new batch of C3H/HeJ mice, only mice that began losing hair were selected. These mice were separated into two groups: control group (no CVT-E002 in diet) and treatment group (fed 80 mg/day of CVT-E002). The CVT-E002 was combined with chow and fed to the mice in the treatment group; while chow without CVT-E002 was fed to the control group. All of the mice in the control group went on to lose all of their body hair. All of the mice (n=14) in the treatment group had complete fur regrowth in 18 weeks. One representative example is shown in FIG. 1.

In addition to regaining their hair, other abnormal behaviours (incessant scratching, inactivity) and low body weight in mice with alopecia areata were reversed by CVT-E002 after 1-2 weeks of treatment. No side effects were observed in any of the mice in the treatment group.

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

### REFERENCES

[1] Monograph: Ginseng, American. http://webprod.hc-sc.gc.ca/nhpid-bdipsn/monoReq.do?id=29&lang=eng, (accessed Nov. 1, 2010).

[2] A. Alkhalifah, A. Alsantali, E. Wang, K. J. McElwee, J. Shapiro, Alopecia areata update: part I. Clinical picture, histopathology, and pathogenesis. J. Am. Acad. Dermatol. 62 (2010) 177-188.

[3] A. Alkhalifah, A. Alsantali, E. Wang, K. J. McElwee, J. Shapiro, Alopecia areata update: part II. Treatment. J. Am. Acad. Dermatol. 62 (2010) 191-202.

[4] L. Kos, J. Conlon, An update on alopecia areata. Curr. Opin. Pediatr. 21 (2009) 475-480.

[5] J. P. Sundberg, W. R. Cordy, L. E. King, Jr., Alopecia areata in aging C3H/HeJ mice. J. Invest Dermatol. 102 (1994) 847-856.

1. A method of preventing or treating alopecia areata in a subject in need of such treatment, the method comprising administering to the subject an effective amount of a ginseng extract having a carbohydrate content comprising about 0.2-5.5 mol % rhamnose, about 9-25 mol % galacturonic acid, about 35-65 mol % glucose, about 8-22 mol % galactose, and about 8-22 mol % arabinose.

2. The method according to claim 1, wherein the ginseng extract has a carbohydrate content comprising about 0.5-5 mol % rhamnose, about 11-22 mol % galacturonic acid, about 40-60 mol % glucose, about 10-19 mol % galactose and about 11-19 mol % arabinose.

3. The method according to claim 1, wherein the ginseng extract is administered at a dose of 1-1000 mg/kg body weight.

4. The method according to claim 1, wherein the ginseng extract is substantially identical to CVT-E002.

5. The method according to claim 1, wherein the ginseng extract is CVT-E002.

6. The method according to claim 1, wherein the ginseng extract is administered orally.

7. The method according to claim 6, wherein the ginseng extract is formulated as a food, nutraceutical or supplement.

8. The method according to claim 1, wherein the ginseng extract is administered topically.

9. (canceled)

10. (canceled)

11. (canceled)

12. (canceled)

13. (canceled)

14. (canceled)

15. (canceled)

16. (canceled)

17. (canceled)

18. A method of preventing or treating alopecia areata in a subject in need of such treatment comprising administering to the subject a ginseng extract comprising ginsenosides Rg1, Re, Rb1, Rd and Rc and from about 60 to about 70% (w/w) carbohydrates.

19. (canceled)

20. (canceled)

21. The method according to claim 18, wherein the ginseng extract is administered orally.

22. The method according to claim 21, wherein the ginseng extract is formulated as a food, nutraceutical or supplement.

23. The method according to claim 18, wherein the ginseng extract is administered topically.

24. The method according to claim 2, wherein the ginseng extract is administered at a dose of 1-1000 mg/kg body weight.

25. The method according to claim 24, wherein the ginseng extract is substantially identical to CVT-E002.

26. The method according to claim 25, wherein the ginseng extract is CVT-E002.

27. The method according to claim 25, wherein the ginseng extract is administered orally.

28. The method according to claim 27, wherein the ginseng extract is formulated as a food, nutraceutical or supplement.

29. The method according to claim 25, wherein the ginseng extract is administered topically.
